(19) United States
(12) Patent Application Publication
(10) Pub. No.: US 2009/0050708 A1
(43) Pub. Date: Feb. 26, 2009

(54) Wickable wearing apparel system containing bait and/or cover scent and method of use

(76) Inventor: Kenneth Gregory Hood, Clarksdale, MS (US)

Correspondence Address: CLARK & BRODY, 1090 VERMONT AVENUE, NW, SUITE 250, WASHINGTON, DC, 20005, US

(21) Appl. No.: 12/222,742

(22) Filed: Aug. 15, 2008

Related U.S. Application Data
(60) Provisional application No. 60/935,483, filed on Aug. 15, 2007.

(51) Int. Cl. A61L 9/00; A61L 9/01; A41D 1/00; A01N 25/06

(52) U.S. Cl. 239/36; 424/76.1; 424/84; 2/69

(57) ABSTRACT

A system for dispersing scent for hunting comprises a material formed into an article of wearing apparel. The material has an inner layer and an outer layer, the inner layer having a looped structure and the outer layer having a tight structure with perforations. The inner layer is adapted to receive perspiration from a user donning the wearing apparel and wick the perspiration through the perforations in the outer layer using body heat from the user. The material also includes an activator containing the scent, the activator contained in the outer layer, the scent being dispersed from the outer layer as a result of body heat emanating from the user for either covering the hunter's odors or attracting animals to the hunter.

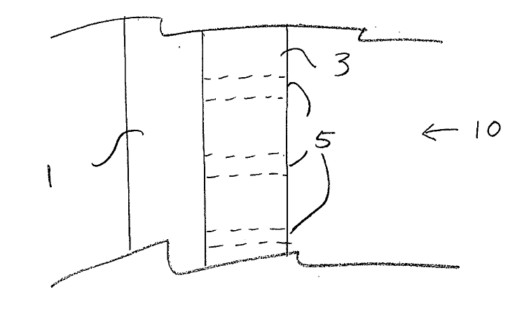

This application claims priority under 35 U.S.C. § 119(e) based on provisional application No. 60/935,483, filed on Aug. 15, 2007.

### FIELD OF INVENTION

The present invention is directed to the incorporation of bait or cover scent in a wickable wearing apparel for use in hunting.

### BACKGROUND ART

The use of various scents or odor eliminators in the sport of hunting are well known in the art. Hunters often time employ certain scents that attract animals, i.e., bait scents. Examples of these scents include corn, persimmon, acorn, honey pears, carrots, sugar beets, acorns, mulberries, licorice, apple, sweet potatoes, turnips, greens, cow peas, blackberries, clover, pecan, peanut butter, alfalfa, oats, chickory, milo, sorghum, molasses, and syrup. Other scents that are employed are typically known as cover scents. These scents are designed to replicate the smell of the environment surrounding the hunter so that the hunter's own odor is masked and the hunter can blend into the surroundings. Examples of cover scents include vanilla, earth aromas, evergreen, sagebrush, and walnut.

It is also well known to use odor eliminators in connection with hunting apparel to eliminate odors emanating from a hunter. One example of this type of odor eliminator is the use of adsorbents such as activated carbon or zeolites integrated into the hunting apparel. In certain instances, the adsorbent is integrated into wearing apparel or other hunting paraphernalia such as backpacks and the like.

United States Published Patent Application No. 2006/0048263 to Walsh, herein incorporated by reference, is directed to garment with hydrophobic arms. One feature of the garment is to wick away perspiration from the underarm portions of the garment and neutralize the odor causing bacteria. In one embodiment, Walsh uses a special fabric for the underarm portion that includes silver-containing fibers. Other options include using fabrics treated with an antimicrobial such as triclosan. A further embodiment includes the use of either an adsorbent such as activated carbon or a chemical that would break down or neutralize the odor.

It is also known to incorporate scents into clothes for hunting purposes. U.S. Pat. No. 6,506,395 to Lillig teaches applying a hedge apple extract onto clothing. U.S. Pat. No. 5,010,589 to Hamilton teaches clothing using fringe material, with the fringe material carrying a scent for hunting.

While various ways have been proposed to incorporate scents into clothing for hunting purposes, these ways still do not disperse the scent in an efficient way. Thus, the effect of the scent, whether it is an attractant for animals or a mask for the hunter's odor, is marginal. That is, the ability to either attract animals or prevent the detection of the hunter using the scent is not maximized in spite of the use of large amounts of the scent or scent holding material.

Therefore, there is a need to improve the dispersion of scents used in hunting over those found in the prior art. The invention responds to this need by providing a system that improves the dispersion of scent for hunting.

### SUMMARY OF THE INVENTION

It is a first object of the invention to provide a system and method of dispersing scent for hunting.

Another object of the invention is to provide a system and method of dispersing scent for hunting that also incorporates an odor eliminator to minimize scents from the hunter.

In satisfaction of the foregoing objects and invention, the invention includes a system for dispersing scent for hunting that comprises a material formed into an article of wearing apparel. The material has an inner layer and an outer layer, the inner layer having a looped structure and the outer layer having a tight structure with perforations. The inner layer is adapted to receive perspiration from a user donning the wearing apparel and wick the perspiration through the perforations in the outer layer using body heat from the user. The material also includes an activator containing the scent, the activator contained in the outer layer, the scent being dispersed from the outer layer as a result of body heat emanating from the user for either covering the hunter's odors or attracting animals to the hunter.

Either of the material or the activator can also include an odor eliminator.

The invention also entails a method of dispersing scent for hunting comprising the steps of providing an article of wearing apparel, the article made from a material having an inner layer and an outer layer, the inner layer having a looped structure and the outer layer having a tight structure with perforations. The inner layer is adapted to receive perspiration from a user donning the wearing apparel and wick the perspiration through the perforations in the outer layer using body heat from the user. The activator containing a scent is incorporated into the outer layer of the article. The article is then donned by a hunter, the hunter generating body heat to dispense the scent from the outer layer to either cover the hunter's odors or attract animals to the hunter.

The step of incorporating the activator can include incorporation of an odor eliminator in the material. Alternatively, the odor eliminator can be incorporated into the activator for material application.

Another aspect of the invention is the activator itself. The activator can include the scent and the odor eliminator for application onto the material.

### BRIEF DESCRIPTION OF THE DRAWINGS

The sole FIGURE depicts a partial cross sectional view of one embodiment of the layered construction-containing material of the invention.

### DESCRIPTION OF THE INVENTION

The invention, in one mode, is a system for maximizing the dispersion of a hunting scent, whether it is a bait scent designed to attract animals or a cover scent intended to mask the hunter's odor. In one embodiment, the system comprises a material that has features which maximizes the ability of the scent to be dispersed, and an activator, which is intended to be incorporated into the material, for either attracting animals or masking the scent of the hunter.

The activator consists of the various cover or bait scents as described above, e.g., pine, cedar, oak, corn, persimmon, acorn, earth or nature scents, etc. Virtually any known bait or cover scent can be employed as an activator of the system. The activator helps the hunter blend in the environment or to bait the desired quarry. The form of the activator can vary, with one form being the scent being dispersed in a base material such as an oil, water, cream, lotion, gel or the like. Preferably, an oil base is used to carry the desired scent. The activator is also preferably made in a sprayable form to facilitate its incorporation into the material.

The material for use as part of the system is a moisture management material. This falls into several different types of knits, jersey knit, double knit, warp knit, tinsel fiber, and is usually 100% polyester. The material comprises at least two layers. One layer, considered to be the wearing apparel outer layer, includes the outer surface of the wearing apparel. This layer is a tight weave of fibers that has small perforated holes in it with an outer surface that is smooth and slick. The second layer, considered to be the inner layer, with its exposed surface intended to be adjacent the wearer has a looped structure of fibers, e.g., is loosely knit. This looped structure is designed to wick or draw moisture from the body and push it to the exterior or outer layer of the clothing.

In a further embodiment, an odor eliminator can be incorporated into the system to eliminate human odor. Examples of odor eliminating ingredients are silver ions, silver-containing fibers, triclosan, quats, activated carbon, zeolites, etc, and combinations thereof. The odor eliminator(s) can be made part of the activator, and then incorporated into the material. Alternatively, the odor eliminators can be incorporated into the material separately from the activator, with the activator added later. For example, silver-containing or activated carbon or zeolite materials such as fibers, powders, or the like could be used when making the material, by either incorporating the odor eliminator into the material to be formed into the fibers, or incorporated the odor eliminator into the formed fibers. The fibers could be manufactured with an odor eliminator as an integral part thereof, or the odor eliminator could be sprayed or otherwise applied to the fibers after their manufacture. If the odor eliminator is manufactured as part of the fibers, it could be in either or both layers. If the odor eliminator is applied to the fibers after manufacture, it is believed that the odor eliminator would be found primarily in the loose weave inner layer, although some would be contained in the tighter weave outer layer given the different construction of the two layers.

The material of the system makes a perfect base for the activator to be applied to the wearing apparel made from the material. In operation, the material is made into a piece of wearing apparel such as a shirt. The activator is applied to the exterior of the shirt. The application of the activator to the shirt can be done in any number of ways. One preferred way is to spray the shirt with a mist applicator with the activator and allow the garment to dry. Other ways include the use of steam and activator.

The loose weave on the inside of the shirt is the wick portion of the shirt. It is designed for moisture to gather at this position. The exterior weave is tight so that it can not retain moisture without having some type of oil base to it. For example if you are wearing the shirt and you begin to sweat the perforated holes allow air to come in and heat from your body to travel out. As you sweat only the loose weave of the interior can gather moisture. As the air comes into the shirt, it dries the moisture that is captured in the loose weave or the heat of the body pushes the moisture to the exterior where it evaporates.

In the embodiment, wherein the system comprises the activator and the material, the base of the activator allow the scent to collect on the exterior of the shirt, i.e., in the outer layer. As body heat attempts to leave the shirt, it is funneled to the small perforated holes in the outer layer. In just a normal wicking shirt, the body heat carries away the perspiration collected in the inner layer of the material, with the perspiration wicking via the outer layer.

In the invention, the warmth of the body stimulates and disperses the scent from the exterior of the shirt. As the heat dissipates from the shirt, it stimulates the scent on the exterior of the shirt (in the outer layer), which dispenses the smell of the bait or the smell that blends in with the hunting area. The scent can escape from the out layer itself and/or through the openings in the outer layer.

In the embodiment wherein the activator is combined with an applied odor eliminator, this acts much like oil and water. The oil base coats the smooth surface of the shirt while the applied odor eliminator tends to travel to the looser thread which happens to be on the inside of the shirt, whereby a majority of the odor eliminator is found in the inner layer.

The bases of the activator allow the scent to collect on the exterior of the shirt while the odor eliminator is attracted to the loose knit of the interior of the shirt. As the odor as well as body heat attempts to leave the shirt, it is funneled to the small perforated holes. There the eliminator in the activator eliminates the odor and the warmth of the body stimulates and disperses the scent from the exterior of the shirt. As the heat dissipates from the shirt, it stimulates the odor on the exterior of the shirt which dispenses the smell of the bait or the smell that blends in with the hunting area.

The sole FIGURE of the application depicts a portion 10 of the layered construction of the material, which as detailed below can take on any number of forms as wearing apparel. The FIGURE illustrates the two layer construction in terms of weave and the primary location of the activator. That is, the loose inner weave 1 is adjacent the outer tight layer weave 3. Throughholes 5 are shown in the outer weave 3. The odor eliminator (not shown) could be included in either weave 1 or 3, with the activator (not shown) preferably contained in the outer layer 3. The weaves of the inner and outer layers are not shown in detail since they are well known in the art.

With the knit of these shirts and the moisture management material, it is possible to both hold the scent and maintain its integrity in the outer layer for a long period of time and also disperse the scent during the hunt. This means that the shirt may be used a number of times before the activator is eventually depleted from the outer layer due to the wicking action of the shirt.

Once the activator is depleted in the shirt, additional activator can be incorporated into the shirt for dispersing during additional hunting excursions. This incorporation can be accomplished by spraying the activator on to the shirt with a mist applicator and allow the garment to dry. When purchasing the wearing apparel, the additional activator can be supplied in the form of a liquid that can then be used when the originally applied activator is depleted.

While a shirt is exemplified as the wearing apparel, other types of wearing apparel could be employed that would be in contact with the hunter's body so that the activator contained in the material of the system can be efficiently dispersed. Examples include pants, shorts, hats, caps, socks, underwear, gloves, scarves, masks, or virtually any material that could contact the hunter's body during the hunt.

While a two layer 100% polyester material is exemplified as part of the system, it is believe that other materials would also function equally as well. Cotton and 50% blend can be stitched in the same formula as the polyester. This allows a tight front and a loose weave on the inside, which is a function of the stitching of the thread. There are hundreds of different material that fall into this category. Other potential materials include 100% cotton and cotton blend, 50/50 as in 50% cotton and 50% polyester, and those including antimicrobial and dry release microbial components. Materials with the antibacterial ingredients woven into the material, for example, silver, triclosan, and other materials, are also believed to be useful materials for the invention.

The activator that is incorporated into the material includes, in one embodiment, effective amounts of the scent and the odor eliminator, and optionally an anti-microbial for the intended purpose of being able to disperse scent without the presence of human odor. In a further more preferred embodiment, the odor eliminator amount would range from 1-3 parts for every 5-8 parts of scent, with an even more preferred ratio of 2 parts odor eliminator to 6 parts scent. The activator is an important aspect of the invention since it is a replenishable feature of the invention. For example, a hunter may purchase a piece of wearing apparel that has the activator without or without the odor eliminator as a part thereof. Once the activator is consumed, the user may spray or soak the shirt with additional activator so that the scent and odor eliminating. functions are again present as part of the wearing apparel. This continued consumption of the activator creates a significant market for activator sales, which far exceeds the sales of the wearing apparel, which will last for a much longer time.

The activator, in any form that can be applied to a material, e.g., a liquid for soaking or spray, gel, paste, etc. The scent part of the activator, bait or cover scent, is an amount effective to be able to either attract animals or mask the odor of the hunter. The odor eliminator portion, when contained as part of the activator for reapplication to a material such as wearing apparel is also in amount effective to eliminate or at least reduce the odors emanating from the hunter.

As such, an invention has been disclosed in terms of preferred embodiments thereof which fulfills each and every one of the objects of the present invention as set forth above and provides a new and improved wickable wearing apparel system containing bait and/or cover scent and method of use.

Of course, various changes, modifications and alterations from the teachings of the present invention may be contemplated by those skilled in the art without departing from the intended spirit and scope thereof. It is intended that the present invention only be limited by the terms of the appended claims.

I claim:

1. A system for dispersing scent for hunting, comprising:
a) a material formed into an article of wearing apparel and having an inner layer and an outer layer, the inner layer having a looped structure and the outer layer having a tight structure with perforations; the inner layer adapted to receive perspiration from a user donning the wearing apparel and wick the perspiration through the perforations in the outer layer using body heat from the user; and
b) an activator containing the scent, the activator contained in the outer layer, the scent being dispersed from the outer layer as a result of body heat emanating from the user for either covering the hunter's odors or attracting animals to the hunter.

2. The system of claim 1, wherein either the material or the activator includes an odor eliminator.

3. A method of dispersing scent for hunting comprising the steps of:
a) providing an article of wearing apparel, the article made from a material having an inner layer and an outer layer, the inner layer having a looped structure and the outer layer having a tight structure with perforations; the inner layer adapted to receive perspiration from a user donning the wearing apparel and wick the perspiration through the perforations in the outer layer using body heat from the user;
b) incorporating an activator containing a scent into the outer layer of the article; and
c) donning the article by a hunter, the hunter generating body heat to dispense the scent from the outer layer to either cover the hunter's odors or attract animals to the hunter.

4. The method of claim 3, further comprising the step of incorporating an odor eliminator into the material or the activator.

5. An activator for use with an article of wearing apparel comprising an effective amount of a scent for either attracting animals or masking an odor of a user of the scent and an effective amount of an odor eliminator for eliminating or reducing odors of the user.

6. The activator of claim 5, wherein the activator is in a liquid sprayable form for application onto the article of wearing apparel.

7. The activator of claim 5, wherein the scent is one of a bait or cover scent.

8. The activator of claim 7, wherein the activator is in a liquid sprayable form for application onto the article of wearing apparel.
